(19) United States
(12) Patent Application Publication
(10) Pub. No.: US 2016/0015392 A1
(43) Pub. Date: Jan. 21, 2016

(54) LUMINAL COMPRESSION DEVICE

(71) Applicant: MAYO FOUNDATION FOR MEDICAL EDUCATION AND RESEARCH, Rochester, MN (US)

(72) Inventor: Matthew T. Gettman, Rochester, MN (US)

(21) Appl. No.: 14/773,484

(22) PCT Filed: Feb. 20, 2014
(86) PCT No.: PCT/US14/17268

§ 371 Date: Sep. 8, 2015

Related U.S. Application Data
(60) Provisional application No. 61/788,685, filed on Mar. 15, 2013.

(51) Int. Cl. A61B 17/122

(52) U.S. Cl. CPC A61B 17/122

(57) ABSTRACT

This document provides devices and methods for the occlusion of bodily lumens. For example, this document provides artificial sphincter devices and methods for treating urinary incontinence.

### CROSS-REFERENCE TO RELATED APPLICATIONS

This application claims the benefit of U.S. Provisional Application Ser. No. 61/788,685, filed Mar. 15, 2013. The disclosure of the prior application is considered part of (and is incorporated by reference in) the disclosure of this application.

### BACKGROUND

1. Technical Field

This document relates to devices and methods for the occlusion of bodily lumens. For example, this document relates to artificial sphincter devices and methods for treating urinary incontinence.

2. Background Information

Urinary incontinence is a very common problem that likely affects millions of people worldwide. Urinary incontinence is the loss of bladder control. The symptoms of urinary incontinence can range from mild leaking during a cough or a laugh, to an uncontrollable emptying of the bladder. Most bladder control problems happen when pelvic floor muscles are too weak or too active.

Various treatments for urinary incontinence have been used. Non-surgical treatments for urinary incontinence have included activities such as losing weight, pelvic floor muscle training, and bladder training. Surgical treatments have included: sling procedures, involving making an incision in the lower abdomen and inserting a sling around the neck of the bladder to support it; urethral bulking agents, to increase the size of the urethral walls thereby allowing the urethra to stay closed with more force; and colposuspension, involving making an incision in the lower abdomen, lifting up the neck of the bladder, and stitching through the walls of the bladder neck to hold it in place.

### SUMMARY

This document provides devices and methods for the occlusion of bodily lumens. For example, this document provides artificial sphincter devices and methods for treating urinary incontinence.

In general, one aspect of this document features a method for constricting a lumen of a mammal. The method comprises: engaging a luminal compression device in the mammal, wherein the luminal compression device is configured to partially surround a conduit that defines the lumen, wherein the luminal compression device includes multiple zones that are configured to independently apply compressive force to the conduit; and applying compressive force to the conduit, wherein the applying comprises individual zones of the multiple zones independently applying compressive force in a successive or sequential pattern.

In various implementations, the mammal may be a human patient. The conduit may be a urethra. In some embodiments of the method, a first compressive force is applied to a first portion and the first portion only of said conduit for a first time period by a first zone of said multiple zones, and, prior to releasing said first compressive force, a second compressive force is applied to a second portion of said conduit by a second zone of said multiple zones. In some embodiments, both the first and second compressive forces are applied concurrently for a second time period. Then first compressive force can be released said while maintaining said second compressive force. Optionally, the first time period can be longer than said second time period. In some embodiments, the luminal compression device may include three or more zones that are configured to independently apply a compressive force to said conduit.

In some implementations, the method for constricting a lumen of a mammal may further comprise deactivating all zones so that no compressive forces are applied to said conduit. Optionally, the deactivating of all zones may comprise using a device configured to wirelessly communicate with said luminal compression device. In some implementations, the method may further comprise programming the luminal compression device to establish a time period that each zone of the multiple zones is activated so as to apply said compressive force to the conduit. In particular implementations, the programming may comprise using a device configured to wirelessly communicate with said luminal compression device. Optionally, the luminal compression device may further comprise one or more motion sensors, and applying a plurality of compressive forces to said conduit may be based on motion of the patient that is detected by said one or more motion sensors.

In another general aspect, this document features a system for constricting a lumen of a mammal. The system comprises: a luminal compression device configured for engagement with the mammal, the luminal compression device being configured to partially surround an outer periphery of a conduit that defines the lumen, and to apply a compressive force on a portion of the outer periphery, wherein the luminal compression device includes multiple zones that are configured to independently apply compressive force to the conduit; and an external controller configured to wirelessly send control commands that are capable of causing the luminal compression device to be activated and deactivated. In this context a compressive force is applied when the luminal compression device is activated, and no compressive force is applied when the luminal compression device is deactivated.

In various implementations, the mammal may be a human patient. The system may further comprise a pH sensor in communication with the luminal compression device. In some embodiments, the system may further comprise one or more motion sensors in communication with the luminal compression device. Optionally, the external controller may be further configured to wirelessly send programming commands that cause the luminal compression device to operate on an ongoing basis in accordance with said programming commands. In some embodiments, the luminal compression device may further comprise fixation components that are configured to prevent migration of the luminal compression device in relation to said patient. In particular embodiments, the luminal compression device may also comprise three or more zones that are configured to independently apply a compressive force to the conduit. In some embodiments, the luminal compression device may be configured to be implanted in the patient.

Particular embodiments of the subject matter described in this document can be implemented to realize one or more of the following advantages. In some embodiments, urinary incontinence can be controlled using the devices and methods provided herein. In some embodiments, an artificial sphincter device can be implanted while minimizing the potential for patient injury, discomfort, and shortening surgical recovery times. In some embodiments, the devices and methods provided herein reduce the risk of ischemia. In some embodiments, the devices provided herein can be wirelessly controlled for enhanced user convenience.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of patient with an implanted artificial sphincter device in accordance with some embodiments provided herein.

FIGS. 2A-2C are illustrations of an exemplary artificial sphincter device in accordance with some embodiments provided herein.

FIG. 3 depicts wireless communications with an implanted artificial sphincter device in accordance with some embodiments provided herein.

Like reference numbers represent corresponding parts throughout.

### DETAILED DESCRIPTION

This document provides devices and methods for the occlusion of bodily lumens. For example, this document provides artificial sphincter devices and methods for treating urinary incontinence. The devices and methods provided herein may also be used to treat other conditions for which the use of a luminal compression device is advantageous. For example, the devices and methods provided herein may have gastric treatment applications (e.g., treatment of gastroesophageal reflux disease) and vascular applications, to provide some additional examples.

The artificial sphincter devices provided herein can be used to compress a bodily lumen. For example, the artificial sphincter devices provided herein can be used to compress a urethra. By compressing the urethra, urinary continence can be treated and controlled.

The artificial sphincter devices provided herein can be used to compress the urethra in both an intracorporeal and extracorporeal context. For example, the artificial sphincter devices provided herein can be used intracorporeally by implanting the device in a patient so that the device can compress the urethra near the base of the bladder. In other cases, the artificial sphincter devices provided herein can be used extracorporeally by using the device to compress the urethra within a penis.

In some embodiments, the artificial sphincter devices provided herein can eliminate or reduce the incidence of ischemia that may be associated with other artificial sphincter devices that compress the urethra, thereby restricting the blood flow in the urethral tissue. In some embodiments, the artificial sphincter devices provided herein use intermittent compression at multiple occlusion zones (focal points) on the urethra to prevent causation of ischemia in the urethra. Further, in some embodiments the artificial sphincter devices provided herein provide partial rather than full circumferential compressions, thereby further reducing potential causation of ischemia in the urethra.

In some embodiments, the artificial sphincter devices provided herein can be implanted using procedures that can reduce patient risks, costs, and discomfort. For example, in some the embodiments the artificial sphincter devices provided herein can be implanted without requiring scrotal invasiveness. Further, the artificial sphincter devices provided herein can reduce the risk of inadvertent urethra entry during implantation because, for example, the artificial sphincter devices provided herein need only a partial, rather than full, circumferential surround of the urethra. In some embodiments, the artificial sphincter devices provided herein can include surface coatings that provide advantages such as reducing the risk of patient infections, reducing tissue inflammation, promoting recovery, and promoting tissue ingrowth and endothelialization.

In some embodiments, artificial sphincter devices provided herein utilize wireless communication technology for device programming and for device control. For example, in some embodiments, the devices can be deactivated using a wireless controller device in communication with the artificial sphincter device (in this context “deactivation” of the device refers to relieving the application of compression from the device on the urethra, such as when the user desires to urinate—or to turn off the device during sleep periods). In some cases, the user can carry a fob-like device that can wirelessly communicate with the artificial sphincter device, e.g., to deactivate and reactivate the device. In some cases, a transmitter can be located in the user's bathroom (or public bathrooms) such that the artificial sphincter device is deactivated when near the transmitter, and reactivated when no longer near the transmitter. In some cases, a cell phone signal or SMS text message signal can be received by the artificial sphincter devices to deactivate and reactivate the device. Other modes of wireless communications and other control functions are also envisioned.

In some embodiments, one or more operative parameters of the artificial sphincter devices provided herein are programmable. In some cases, a computing system, such as laptop computer for example, can be used to program particular parameters related to the operations of the artificial sphincter devices. The parameters can include certain time-based aspects of operation. For example, the time duration that a particular zone of occlusion remains in the closed state is programmable in some embodiments. The time duration that successive zones of occlusion overlap each other can be programmable in some embodiments. The sequence that the various zones of occlusion operate in can be programmable in some embodiments.

In some embodiments, wireless communications can be used to program and to calibrate the artificial sphincter devices provided herein. That is, in some embodiments, operative parameters that affect the function of the devices can be programmed using a programming device that wirelessly communicates with the artificial sphincter devices provided herein. This feature allows for convenient on-the-go adjustments of the artificial sphincter devices, with minimal user invasiveness. The adjustments can be used, for example, to improve the device's efficacy, improve patient comfort or convenience, to calibrate the device, and to optimize other factors.

Further additional functional features are included in some embodiments of the artificial sphincter devices and methods provided herein. In some embodiments, fixation components are included to prevent or minimize unintended migration of the device. In some embodiments, a pH sensor is included to provide detection of urine downstream of the device. The pH sensor can be used to signal to the artificial sphincter device to increase compression in some embodiments. In some embodiments, various power sources or supplemental power sourced are included. The power sources can include human kinetic energy sources such as luminal flow generators, kinetic magnetos (movement of appendages) caloric generators (deriving energy from the body's core temperature), and photo-generators (implantable solar cells), to provide a few examples.

Other functional features of the invention would provide for the concept of dynamic sphincter control. Sensors would be included that facilitate dynamic sphincter control. When these sensors are activated, then the above programming could be set with override type functions to further increase compression of the device. The programming could also be set so that the sensors are activated by positional changes (going from supine to standing as an example), or by movement. When the sensors are activated the compression could be increased. This would provide the patient with an additional means for “on demand” compression. In some embodiments, positional change sensors can be derived on the basis of a metallic liquid switch. When the patient is supine, the switch would be in an off position. When the patient moves to an upright position, the metallic fluid in the switch would move by gravity similarly to a dependent position and this would complete the circuit. With the circuit complete, the device would (when appropriately programmed) increase the compression forces in the zone of compression.

Other sensors could be used to detect patient motion. This is beneficial because some patients have increased leakage when they walk or run. Multiple different types of sensors could be used to assess movement. Kinetic motion sensors would be one example. It is also possible that the device could have a microphone as a component. The microphone would hear the repetitive sounds of walking or running and based on the frequency, increased or decreased compression could be enacted. It is also possible, that a combination of sensors could be used to achieve dynamic sphincter compression. While the above types of sensors are particularly described, it is also envisioned that a variety of other types of sensors known in the art could be used for providing dynamic sphincteric compression.

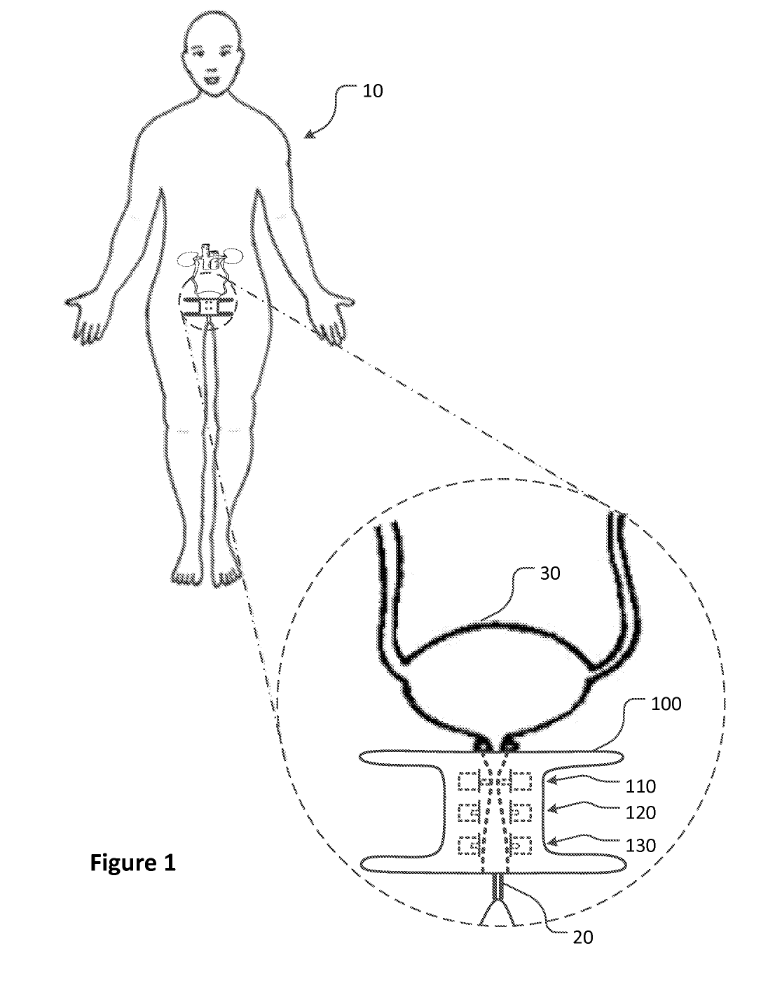

With reference to FIG. 1, a human patient 10 is depicted as having an implanted artificial sphincter device 100 that partially surrounds a urethra 20 at the base of a bladder 30. Patient 10 can be a male or a female, without substantially affecting the description of devices and methods provided herein. For men, the device can be implanted via a longitudinal incision made in the perineum. For women, the device can be implanted via a lower abdominal incision. In men the device can be placed around the bulbar urethra. In women the device can be placed around the bladder neck.

When activated, artificial sphincter device 100 can apply compression to urethra 20 so that the flow of urine from bladder 30, through urethra 20, and to the outside the body of patient 10 is blocked. When deactivated, artificial sphincter device 100 can remove the compression on urethra 20, so that urine can flow from bladder 30, through urethra 20, and to the outside of the body of patient 10.

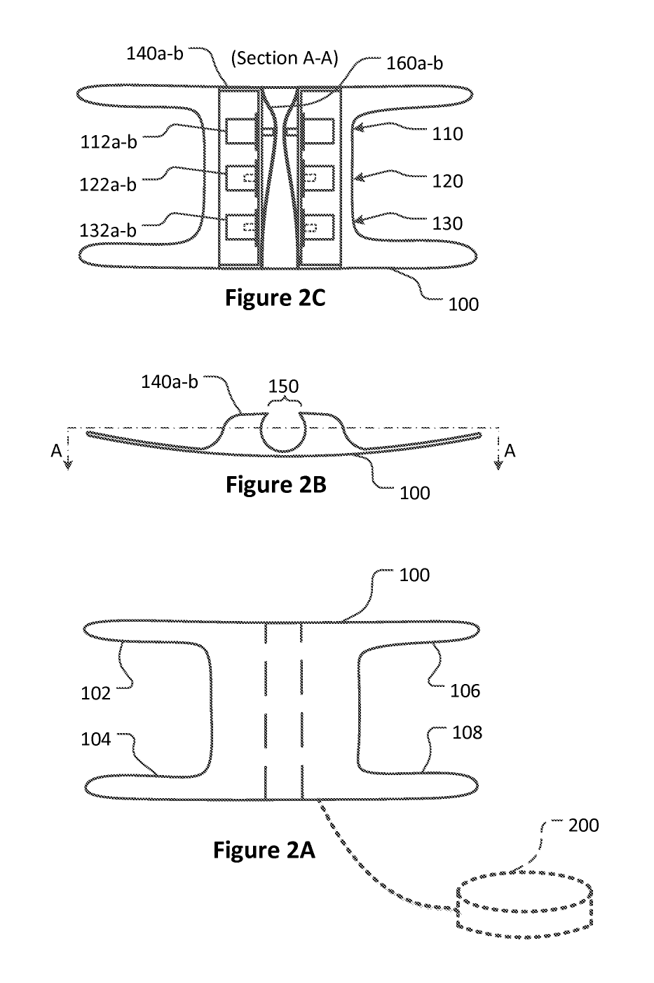

As described further in reference to FIG. 2A below, artificial sphincter device 100 can include multiple occlusion zones. For example, artificial sphincter device 100 includes an upper occlusion zone 110, a middle occlusion zone 120, and a lower occlusion zone 130. In some other embodiments, artificial sphincter devices provided herein can include two occlusion zones, or four occlusion zones, or more than four occlusion zones.

It is also possible that the artificial sphincter device could include the implantation of multiple devices that include one or more occlusion zones. If multiple devices are implanted, a minimum of two devices would need to be implanted. In this manner, one device could be at rest and one working (in turns, alternately) at all times to provide urethral compression and minimize adverse effects of a single zone of constant compression the urethral circumferentially in the same area. When multiple compression devices are implanted, the timing and synchronization of devices would be employed to achieve urinary control. Furthermore, use of single zone occlusion devices, as an example, could be used in conjunction with previously placed failed or poorly functioning hydraulic artificial urinary sphincters.

In some embodiments, artificial sphincter device 100 can operate as follows. In general, at any one time, one occlusion zone is closed, and two occlusion zones are open. In other words, one occlusion zone is actively applying compressive force to urethra 20, and the other two occlusion zones are not applying compressive force to urethra 20. As used herein, “closed” means that the occlusion zone is applying compressive force to the urethra, and “open” means that the occlusion zone is not applying compressive force to the urethra. For example, as shown in FIG. 1 (as best seen in the enlarged view), upper occlusion zone 110 is closed, while middle occlusion zone 120 and lower occlusion zone 130 are open.

After the lapse of a period of time during which upper occlusion zone 110 is closed and middle occlusion zone 120 and lower occlusion zone 130 are open, then one of either middle occlusion zone 120 or lower occlusion zone 130 will close. For the sake of making this example clear, middle zone 120 can be assumed to have closed. At that point in time (for a short period of time), two occlusion zones are closed—upper zone 110 and middle zone 120. There is a short overlap of time during which both occlusion zones 110 and 120 are closed (typically a fraction of a second). Then, upper occlusion zone 110 will open. At that point in time, middle occlusion zone 120 is closed and the other occlusion zones 110 and 130 are open. The purpose of the overlap of time during which both occlusion zones 110 and 120 are closed is to ensure that urethra 20 is always compressed (including, for example, during the transition from upper occlusion zone 110 to middle occlusion zone 120). That way, urine will be prevented from inadvertently flowing.

After another lapse of a period of time during which middle occlusion zone 120 is closed and upper occlusion zone 110 and lower occlusion zone 130 are open, then lower occlusion zone 130 will close. As with the transition between the upper occlusion zone 110 and the middle zone 120, for a short period of time both middle occlusion zone 120 and lower occlusion zone 130 will be closed. Then, middle zone 120 will open and only lower zone 130 will be closed.

In some embodiments, this pattern of cycling between occlusion zones 110, 120, and 130 will continue on as long as artificial sphincter device 100 is activated. One purpose of such cycling is to prevent or reduce the likelihood of ischemia of urethra 20. In general, ischemia can occur when blood flow is cut off from being supplied to live tissues that need oxygenated blood for vitality. If an artificial sphincter compresses the same position on urethra 20 for long enough, ischemia of urethra 20 may result because some urethral tissue may not get the oxygen that it needs. However, by cycling the application of compressive forces between occlusion zones 110, 120, and 130, blood flow blockages that result in ischemia can be prevented, or made substantially less likely.

In some embodiments, various parameters involved in the operations described above can be programmed for artificial sphincter device 100. For example, in some embodiments the duration of time during which a particular occlusion zone is closed can be established by programming the artificial sphincter device 100. In some cases, the duration during which a particular occlusion zone is closed may be about 1 second. In some cases, the duration may be about 0.5 seconds. In some cases, the duration may be about 1.5 seconds, about 2.0 seconds, about 2.5 seconds, about 3 seconds, or longer than 3 seconds. The overlap time during which two occlusion zones are closed can be programmable in some embodiments. For example, in some cases the overlap time may be about 0.1 seconds, about 0.2 seconds, about 0.3 seconds, about 0.4 seconds, about 0.5 seconds, or more than about 0.5 seconds. In some embodiments, the sequence order (pattern) of occlusion zones 110, 120, and 130 can be programmed. In other words, in some cases, the occlusion zones may operate in a sequence like 110-120-130-110-120-130 and so on. In some cases, the occlusion zones may operate in a sequence like 110-130-120-110-130-120 and so on. Other types of sequences can also be used. In some embodiments, various other parameters can also be established and controlled by programming artificial sphincter device 100.

In reference to FIGS. 2A-2C, artificial sphincter 100 is shown in front, top, and rear-sectional views. That is, FIG. 2A is a front view of artificial sphincter 100, FIG. 2B is a top view of artificial sphincter 100, and FIG. 2C is a rear-sectional view of artificial sphincter 100 (along section A-A of FIG. 2B). Section A-A allows a view of an internal portion of artificial sphincter 100, thereby exposing the actuators of the occlusion zones 110, 120, and 130. It is disclosed that the number of occlusion zones for a given device could vary from one or more. While three compression zones are shown as an example, in some cases, more than three zones can be beneficial to achieve the intended clinical goal.

In reference to FIG. 2A, artificial sphincter device 100 with wings 102, 104, 106, and 108 is illustrated. An optional supplemental power source 200 is also illustrated.

In some embodiments, wings 102, 104, 106, and 108 can be used to stabilize and/or anchor artificial sphincter device 100 within the anatomy of a user, e.g., to prevent unintended migration of artificial sphincter device 100. In general, in some embodiments, fewer than four wings are used. For example, in some embodiments, one, two, or three wings are used. Wings 102, 104, 106, and 108 are flexible to conform to the user's anatomy. In some cases, wings 102, 104, 106, and 108 are sutured, clipped, or anchored to the anatomy of the user. In some cases, wings 102, 104, 106, and 108 are adhered to the anatomy of the user using a surgical adhesive. In some cases, wings 102, 104, 106, and 108 can be anchored to the anatomy of the user using barbs, hooks, helical anchors, protrusions, etc., on wings 102, 104, 106, and 108. In some cases, other suitable means of anchoring or attachment are used. In some cases, just the presence of wings 102, 104, 106, and 108 in contact with the anatomy of the user (without further attachment techniques) will stabilize the location of artificial sphincter device 100 within the user.

In some embodiments, the surfaces of artificial sphincter device 100 may be modified with chemical coatings that promote one or more of endothelial cell attachment, endothelial cell migration, endothelial cell proliferation, resistance to bacterial growth, and resistance to thrombosis. In some embodiments, the surfaces of artificial sphincter device 100 may be modified with covalently attached heparin or impregnated with one or more drug substances that are released in situ to promote wound healing or reduce tissue inflammation. In some embodiments, the drug may be a corticosteroid, a human growth factor, an anti-mitotic agent, an antithrombotic agent, or dexamethasone sodium phosphate, to provide a few examples.

In some embodiments, an optional supplemental power source 200 may be included. In some cases, optional supplemental power source 200 can be implanted in a pocket under the user's skin. Optional supplemental power source 200 can provide additional runtime of artificial sphincter device 100 between replacements of the battery of artificial sphincter device 100. In some cases, an inductive charging technique of the battery of artificial sphincter device 100 and/or of optional supplemental power source 200 is used.

With reference to FIG. 2B, artificial sphincter device 100 with two actuator housings 140a-b and an urethral opening 150 is depicted. As described further in reference to FIG. 2C below, actuator housings 140a-b are liquid sealed compartments that contain the mechanisms that comprise the occlusive zones 110, 120, and 130.

Urethral opening 150 is a C-shaped (or horseshoe-shaped) clearance slot or opening in the profile of artificial sphincter device 100. Artificial sphincter device 100 may be available in different sizes, including different sizes of urethral opening 150 to accommodate different sizes of users. Urethral opening 150 will partially surround the user's urethra when artificial sphincter device 100 is installed. The C-shape of urethral opening 150 facilitates the installation (and removal) of artificial sphincter device 100. That is, because urethral opening 150 only partially surrounds the urethra, artificial sphincter device 100 can be installed in less invasive manner in comparison to other artificial sphincter devices that fully surround the urethra.

With reference to FIG. 2C, artificial sphincter device 100 is shown in a cross-sectional view to expose actuators 112a-b, 122a-b, and 132a-b contained within actuator housings 140a-b. In some embodiments, each occlusion zone 110, 120, and 130 includes two actuators 112a-b, 122a-b, and 132a-b respectively.

In general, actuators 112a-b, 122a-b, and 132a-b operate in pairs. That is 112a and 112b operate in concert with each other—as shown in FIG. 2C wherein actuators 112a and 112b are both closed. Similarly, actuators 122a and 122b operate in concert with each other. And, actuators 132a and 132b operate in concert with each other. However, in some embodiments, rather than pairs of actuators that operate in concert with each other, single actuators are used. In some embodiments, three or more actuators are used that in concert with each other.

A variety of type of actuators 112a-b, 122a-b, and 132a-b can be used. In some embodiments, linear electrical solenoid devices (with pistons) can be used for the actuators 112a-b, 122a-b, and 132a-b. In some embodiments, rotary actuators with arms (e.g., similar to the flippers of a pinball machine) can be used. In some embodiments, rotary cams can be used. In some embodiments, roller balls can be used. In some embodiments, the actuators 112a-b, 122a-b, and 132a-b can gear driven. In some embodiments, the actuators 112a-b, 122a-b, and 132a-b can be driven by a flexible timing belt. In some embodiments, the actuators 112a-b, 122a-b, and 132a-b can be driven by flexible driveshafts. In some embodiments, other suitable means of actuation can be used.

In some embodiments, artificial sphincter device 100 includes a flexible covering 160a-b. In general, flexible covering 160a-b provides a way for occlusion zones 110, 120 and 130 to be movable, while maintaining a liquid sealed artificial sphincter device 100. Flexible covering 160a-b can be a flexible, elastic, a durable material. In some embodiments, flexible covering 106a-b is a fluoropolymer (e.g., PTFE or ePTFE), a polyester, a silicone, a urethane, or another suitable flexible biocompatible material. In some embodiments, the flexible covering 106a-b may be impregnated with one or more drug substances that are released in situ to promote wound healing, reduce the chance of infection, or reduce tissue inflammation.

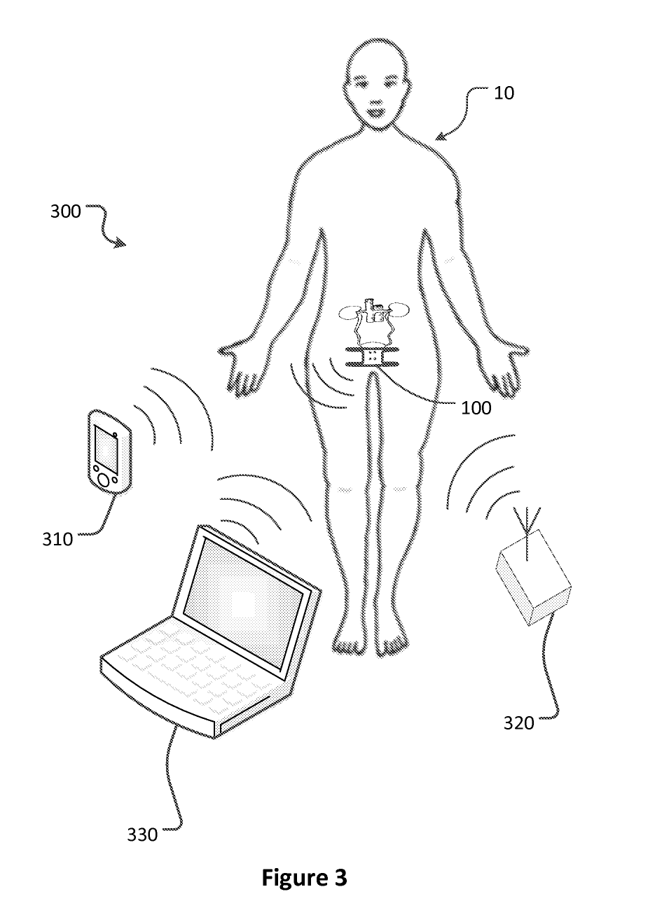

With reference to FIG. 3, artificial sphincter device 100 is depicted in wireless communications 300 with a variety of devices. In general, artificial sphincter device 100 can communicate using a variety of wireless technologies. For example, in some cases, radio frequency (“RF”) communications can be used. In some cases, Bluetooth, infrared, ultrasound, cell phone signals, SMS text messages, and other various suitable wireless modes of device communication can be used for wireless communications between artificial sphincter device 100 and external controllers/programmers.

In some embodiments, a user device 310 can be used to wirelessly control artificial sphincter device 100. In some cases, user device 310 can be a key-fob type of device. In some cases, user device 310 can be a mobile communication device such as a cell phone, PDA, tablet computer, wearable computer, MP3 player, and the like. In general, user device 310 can activate and deactivate (open and close) and otherwise control artificial sphincter device 100 based on commands entered to user device 310 and wirelessly transmitted to artificial sphincter device 100. In some cases, cell phone and SMS text message signals can be the mode of communication. This feature would allow the user to easily control the amount of urethral compression during off peak times such as sleeping. In doing so, the blood flow to the urethra would be maximized in the off peak times. This would work to substantially reduce the amount of urethral atrophy that can occur over time with existing artificial sphincter devices.

In some embodiments, a transmitter 320 can wirelessly control artificial sphincter device 100. Transmitter 320, may be located in a bathroom for example. In some cases, transmitter 320 provides a short-range signal to deactivate (open) artificial sphincter device 100. Accordingly, when user 10 is within a short-range (about 3 feet in some embodiments, or more or less in other embodiments), artificial sphincter device 100 will receive the wireless signal from transmitter 320 to deactivate. The user may then urinate without interference from artificial sphincter device 100. After leaving the proximity of transmitter 320, artificial sphincter device 100 will reactivate because of no longer being within range of the signal from transmitter 320.

In some embodiments, a computer 330 can wirelessly control artificial sphincter device 100. Further, in some embodiments computer 330 can wirelessly program artificial sphincter device 100. As described herein, various operative parameters can be programmed to control the operations of artificial sphincter device 100. Computer 330 can wirelessly perform such programming.

In some embodiments, a variety of other types of devices can similarly wirelessly communicate with artificial sphincter device 100. Such wireless devices can provide control over artificial sphincter device 100 and programming of artificial sphincter device 100. In some cases, wireless inductive charging of the battery of artificial sphincter device 100 can also be performed.

The concepts described above could also be adapted for use as an external compression device of the penis. External compression devices (e.g. Cunningham clamps) are used by some men as a non-surgical means to treat severe urinary incontinence. These devices are suitable for men that are not considered surgical candidates, or for men that will not accept the risks of surgery. External compression devices are also the basis of condom catheters that are used by some patients with neurogenic bladders. A limitation of all such external compression devices is that the compression is in a single area. Such devices can also tend to result in adverse skin irritation. For the described external application, a doughnut-type ring device can be used. In similar fashion as the internal device, zones of compression would be present with in the doughnut. These features would provide a clamping effect on the penis. The embodiments of the external compression device provided herein can compress the urethra and prevent urinary leakage. With deactivation of the external compression device, the urethral compression would be released and urine would be allowed to flow.

In another embodiment, the external compression device could be used to facilitate continuous urine drainage, and include a feature to attach to a urinary catheter or other urine collection device. In essence, the external compression device would hold the urinary catheter in place.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for constricting a lumen of a human patient, wherein said method comprises:
(a) engaging a luminal compression device with said patient, wherein said engaging comprises said luminal compression device partially surrounding a conduit that defines said lumen, wherein said luminal compression device includes multiple zones that are configured to independently apply a compressive force to said conduit; and
(b) applying a plurality of compressive forces to said conduit, wherein said applying comprises individual zones of said multiple zones independently applying compressive force sequentially.

2. The method of claim 1, wherein said conduit is a urethra.

3. The method of claim 1, wherein a first compressive force is applied to a first portion and the first portion only of said conduit for a first time period by a first zone of said multiple zones, and, prior to releasing said first compressive force, applying a second compressive force to a second portion of said conduit by a second zone of said multiple zones, wherein both the first and second compressive forces are applied concurrently for a second time period, and then releasing said first compressive force while maintaining said second compressive force.

4. The method of claim 3, wherein said first time period is longer than said second time period.

5. The method of claim 1, wherein said luminal compression device includes three or more zones that are configured to independently apply a compressive force to said conduit.

6. The method of claim 1, further comprising deactivating all zones so that no compressive forces are applied to said conduit.

7. The method of claim 6, wherein said deactivating comprises using a device configured to wirelessly communicate with said luminal compression device.

8. The method of claim 1, further comprising programming said luminal compression device to establish a time period that each zone of the multiple zones is activated so as to apply said compressive force to said conduit.

9. The method of claim 8, wherein said programming comprises using a device configured to wirelessly communicate with said luminal compression device.

10. The method of claim 1, wherein said luminal compression device further comprises one or more motion sensors, and wherein said applying a plurality of compressive forces to said conduit, is based on motion of the patient that is detected by said one or more motion sensors.

11. A system for constricting a lumen of a human patient, said system comprising:
(a) a luminal compression device configured for engaging with said patient, said luminal compression device configured to partially surround an outer periphery of a conduit that defines said lumen, and to apply a compressive force on a portion of said outer periphery, wherein said luminal compression device includes multiple zones that are configured to independently apply compressive force to said conduit; and
(b) an external controller configured to wirelessly send control commands that are capable of causing said luminal compression device to be activated and deactivated, wherein a compressive force is applied when said luminal compression device is activated, and wherein no compressive force is applied when said luminal compression device is deactivated.

12. The system of claim 11, further comprising a pH sensor in communication with said luminal compression device.

13. The system of claim 11, further comprising one or more motion sensors in communication with said luminal compression device.

14. The system of claim 11, wherein said external controller is further configured to wirelessly send programming commands that cause said luminal compression device to operate on an ongoing basis in accordance with said programming commands.

15. The system of claim 11, wherein said luminal compression device comprises fixation components that are configured to prevent migration of said luminal compression device in relation to said patient.

16. The system of claim 11, wherein said luminal compression device comprises three or more zones that are configured to independently apply a compressive force to said conduit.

17. The system of claim 11, wherein said luminal compression device is configured to be implanted in said patient.
